(19) United States
(12) Patent Application Publication
(10) Pub. No.: US 2025/0366736 A1
(43) Pub. Date: Dec. 4, 2025

(54) APPARATUS, AND SYSTEM FOR AN UPPER EXTREMITY MAGNETIC RESONANCE DYNAMOMETER WITH REAL-TIME FEEDBACK

(71) Applicant: UNIVERSITY OF FLORIDA RESEARCH FOUNDATION, INCORPORATED, Gainesville, FL (US)

(72) Inventors: Kinyata J. COOPER, Gainesville, FL (US); Scott A. BANKS, Gainesville, FL (US); Federico POZZI, Gainesville, FL (US); Sean FORBES, Gainesville, FL (US)

(21) Appl. No.: 19/208,210

(22) Filed: May 14, 2025

Related U.S. Application Data
(60) Provisional application No. 63/654,621, filed on May 31, 2024.

(51) Int. Cl. A61B 5/11; A61B 5/00

(52) U.S. Cl. CPC A61B 5/1107; A61B 5/742; A61B 5/6823; A61B 5/6825; A61B 5/6831; A61B 2562/0252

(57) ABSTRACT

Provided herein is an apparatus and system for an upper extremity magnetic resonance dynamometer able to provide real time feedback regarding physiology of a patient. A system can include: a magnetic resonance dynamometer; an analog-to-digital converter; and a computer, wherein the magnetic resonance dynamometer is configured to measure a force exerted by a patient during a magnetic resonance imaging procedure, and wherein the computer provides for a graphical indication of the force exerted in real time during the magnetic resonance imaging procedure. The magnetic resonance dynamometer of some embodiments is an upper extremity magnetic resonance dynamometer configured to measure the abduction force exerted by the patient from an upper extremity.

### STATEMENT OF GOVERNMENT SUPPORT

This invention was made with government support under Grant No. R21 AR077231, awarded by the National Institutes of Health. The government has certain rights in the invention.

### TECHNOLOGICAL FIELD

An example embodiment of the present disclosure relates to a method, apparatus, and system for a magnetic resonance dynamometer, and more specifically, an upper extremity magnetic resonance dynamometer able to provide real time feedback regarding physiology of a patient.

### BACKGROUND

Various types of bodily injuries can lead to loss of function or degraded muscle performance even after the injury is fully healed. Tendons, muscles, and scar tissue can alter the anatomy of a region of the body after an injury and through healing. Further, vascularity can be altered after an injury resulting in a change in blood flow to an area of the body. The degenerative processes after an injury, such as a rotator cuff tear, can include fat accumulation (around and within the muscle), altered vascularity, and reduction of the functional contractile mass. For example, vascularity can be altered after a rotator cuff tear in the shoulder. This is known based on historical studies that have taken tissue samples in a somewhat invasive procedure. The extent to which this occurs is not widely understood as the invasiveness of the gathering of tissue samples is undesirable, especially if sampled over time during healing after an injury.

### BRIEF SUMMARY

Embodiments of the present disclosure provide a method, apparatus, and system for an upper extremity magnetic resonance dynamometer able to provide real time feedback regarding physiology of a patient. Embodiments include an apparatus including a first strap configured to be attached to a patient proximate a first location; a second strap configured to be attached to the patient proximate a second location; and a load cell connected between the first strap and the second strap, wherein the load cell is configured to measure a force exerted between the first location of the patient and the second location of the patient.

According to some embodiments, the apparatus is magnetic resonance compatible. The load cell of example embodiments is magnetic resonance compatible. According to some embodiments the first strap is configured to be attached to the patient proximate a waist of the patient, wherein the second strap is configured to be attached proximate a wrist of the patient. According to certain embodiments a force exerted between the waist of the patient and the wrist of the patient comprises a contraction of at least one muscle in a shoulder associated with an abduction movement.

According to some embodiments the load cell is connected to the first strap with a first fastener and to the second strap with a second fastener, wherein at least one of the first fastener and the second fastener are removably connected to the load cell. The first fastener and the second fastener of some embodiments each permit rotation of the load cell relative to the first strap and the second strap. The apparatus of some embodiments further includes a first washer and a second washer, where the first strap is disposed between the load cell and the first washer and where the second strap is disposed between the load cell and the second washer.

Embodiments provided herein include a system including: a magnetic resonance dynamometer; an analog to digital converter; and a computer, wherein the magnetic resonance dynamometer is configured to measure a force exerted by a patient during a magnetic resonance imaging procedure, and wherein the computer provides for a graphical indication of the force exerted in real time during the magnetic resonance imaging procedure. The magnetic resonance dynamometer of some embodiments is an upper extremity magnetic resonance dynamometer configured to measure the force exerted by the patient upper extremity.

The magnetic resonance dynamometer of some embodiments includes: a first strap configured to be attached to a patient proximate a first location; a second strap configured to be attached to the patient proximate a second location; and a load cell connected between the first strap and the second strap, wherein the load cell is configured to measure the force exerted between the first location of the patient and the second location of the patient. The magnetic resonance dynamometer of an example embodiment is magnetic resonance imaging compatible. The load cell of an example embodiment is magnetic resonance imaging compatible.

According to some embodiments the first strap is configured to be attached to the patient proximate a waist of the patient, where the second strap is configured to be attached proximate a wrist of the patient. According to certain embodiments the force exerted between the waist of the patient and the wrist of the patient comprises a contraction of at least one muscle in a shoulder associated with an intended movement. The load cell of some embodiments is connected to the first strap with a first fastener and to the second strap with a second fastener, wherein at least one of the first fastener and the second fastener are removably connected to the load cell. According to certain embodiments the first fastener and the second fastener each permit rotation of the load cell relative to the first strap and the second strap. The system of some embodiments further includes a first washer and a second washer, where the first strap is disposed between the load cell and the first washer and where the second strap is disposed between the load cell and the second washer. The system of some embodiments includes a display, wherein the display is configured to display a force signal as the patient exerts force in real time during the magnetic resonance imaging procedure.

### BRIEF DESCRIPTION OF THE DRAWINGS

Having thus described certain example embodiments in general terms, reference will hereinafter be made to the accompanying drawings, which are not necessarily drawn to scale, and wherein:

FIG. 1 illustrates a system for implementing an upper extremity magnetic resonance dynamometer able to provide real time feedback regarding physiology of a patient according to an example embodiment of the present disclosure;

FIG. 2 illustrates an upper extremity magnetic resonance dynamometer according to an example embodiment of the present disclosure;

FIG. 3 illustrates a detail view of an upper extremity magnetic resonance dynamometer according to an example embodiment of the present disclosure; and

FIG. 4 illustrates a series of displays for a force target for a participant and a time series force trace displaying target force and generated shoulder force, according to an example embodiment of the present disclosure.

### DETAILED DESCRIPTION

Some example embodiments of the present disclosure will now be described more fully hereinafter with reference to the accompanying drawings, in which some, but not all embodiments of the invention are shown. Indeed, various embodiments of the disclosure may be embodied in many different forms and should not be construed as limited to the example embodiments set forth herein; rather, these example embodiments are provided so that this disclosure will satisfy applicable legal requirements. Like reference numerals refer to like elements throughout.

Rotator cuff tears are a common cause of pain and disability among adults. Histological studies demonstrate that the degenerative process following a rotator cuff tear disrupts the vascularity and energetics of the shoulder muscles. Analysis of the vascularity and energetics of the shoulder muscles is challenging given current techniques, particularly non-invasive techniques. Understanding the vascularity can inform medical practitioners of a patient's recovery status and identify issues that are best addressed early in the recovery period.

One non-invasive technique for analysis of muscles is Quantitative Magnetic Resonance (QMR). This technique can measure fat mass, lean mass, and total body water along with providing more specific bone structure parameters than dual-energy X-ray absorptiometry (DEXA). Thus, QMR markers offer a promising avenue to assess the muscle non-invasively. Post- contractile MR imaging-based blood oxygen level-dependent (MRI-BOLD) response is a marker that reflects the balance of oxygen delivery and utilization in skeletal muscle. Inadequate delivery of oxygen to skeletal muscles can impede healing and recovery and contribute to altered muscle energetic status. 31Phosphorus MR spectroscopy (31P-MRS) can measure ratios of high energy phosphates (i.e., inorganic phosphate, phosphocreatine, and ATP), which have been associated with muscle health and pathology.

Focusing on mechanisms of muscle degeneration (rather than structural) after rotator cuff tears can advance the understanding of the ability of rotator cuff muscle to heal and respond to treatment, offering value for future studies assessing healing capacity and novel therapeutic interventions. However, muscle movement/contraction under controlled circumstances is necessary to understand a level of muscle degeneration.

Functional magnetic resonance imaging (MRI) can be used to study muscles as they undergo contractions. Muscle energetics and vascularity using a functional MRI can provide insight into the degree of muscle degeneration in a patient. While muscle contraction during an MRI can provide insight, understanding a degree of muscle contraction is challenging. Embodiments described herein provide an upper extremity magnetic resonance dynamometer with real-time feedback. Embodiments are able to provide feedback regarding the force exerted by a muscle during a dynamic MRI that can be used to understand the degree of muscle contraction and enable better analysis of the vascularity and energetics in a muscle.

Vascularity of a shoulder is altered after a rotator cuff tear injury. Measuring vascularity non-invasively improves the understanding of a degree of muscle degeneration and can inform the process for rehabilitating the muscle. Embodiments described herein enable measurement of post-contractile blood-oxygen level dependency (MRI-BOLD) in shoulder muscles during an MRI exam. This provides a measure of blood delivery and usage during a contraction. Embodiments include an MR-compatible device that not only allows a shoulder isometric contraction but measures a degree of force exerted by the contraction.

Clinical outcomes of patients with rotator cuff tears have not improved in decades. Rotator cuff tears have conventionally been treated as a pathoanatomical disorder; the tendon tear is the origin of the symptoms; fixing the tear will cure the symptoms. Decades of research have dramatically improved the pathoanatomical and biomechanical understanding of the rotator cuff without associated improvements in clinical outcomes. Rotator cuff tear-related pain symptoms are complex. Thus, applicant has considered an alternative hypothesis: the brain can modulate the pain symptoms. The premise of this hypothesis lies with findings in other musculoskeletal disorders. Embodiments described herein enable assessment of rotator cuff tears from pain and neuroscience perspectives. Understanding the mechanisms that mitigate or aggravate symptoms is essential for developing effective treatments and optimize outcomes for potentially 4.5 million people seeking care for rotator cuff tears each year in the U.S. alone.

A motor-task functional neuroimaging paradigm that directly engages the painful shoulder can reveal how structural disruption and symptoms interfere with movements. Pain alters peripheral motor activity independently from the mechanical impact of rotator cuff tears. Individuals with pain can shift biomechanical demands away from painful areas suggesting the existence of pain-mitigating volitional alterations in motor execution. Embodiments described herein employ a motor-task paradigm to acquire functional neuroimaging while participants perform submaximal shoulder abduction contractions. This task directly engages the painful shoulder using a magnetic resonance compatible shoulder dynamometer such that an MRI can be performed during shoulder contractions.

Employing embodiments of the MRI compatible shoulder dynamometer described herein can determine how the central nervous system influences symptoms to explain the heterogenous clinical presentation of the variable response to clinical care of patients with rotator cuff tears. Rotator cuff structural damage (e.g., tear size, retraction, arthropathy, etc.) and pain severity are not necessarily related. Embodiments enable determination of the role of the central nervous system in rotator cuff tears trough functional neuroimaging. Patients with massive and irreparable rotator cuff tears (end-stage disease) show adaptations in cortical regions associated with chronic pain and body motion perception. These indications support the proposed motor-task neuroimaging paradigm that can be assessed employing embodiments of the system described herein.

Understanding the interaction between the central nervous system, pain symptoms, and recovery is necessary before pathoanatomically driven clinical decisions can be challenged to enable precision medicine for rotator cuff tears. Clinical decisions are generally based primarily on pathoanatomical information, yet rotator cuff structural changes do not fully explain the severity of pain symptoms or predict recovery. Embodiments described herein aim to bridge a critical gap by determining if the central nervous system plays a larger role in symptom severity and recovery than conventionally believed.

Embodiments enable the comprehensive identification of specific pain phenotypes within rotator cuff tears. The motor-task functional neuroimaging paradigm is innovative as the motor task (submaximal isometric abduction contraction) mimics a commonly painful activity for patients with rotator cuff tears (i.e., lifting the arm).

Using advanced magnetic resonance imaging and spectroscopy using a scanner with simultaneous multiple-splice, phased array acceleration, high-resolution echo-planar imaging, embodiments acquire functional MRI while participants perform isometric shoulder abduction contractions. These tasks directly engage the painful shoulder, the body area associated with the primary clinical symptoms. Three dimensional T1 brain images can be acquired to register and localize functional images.

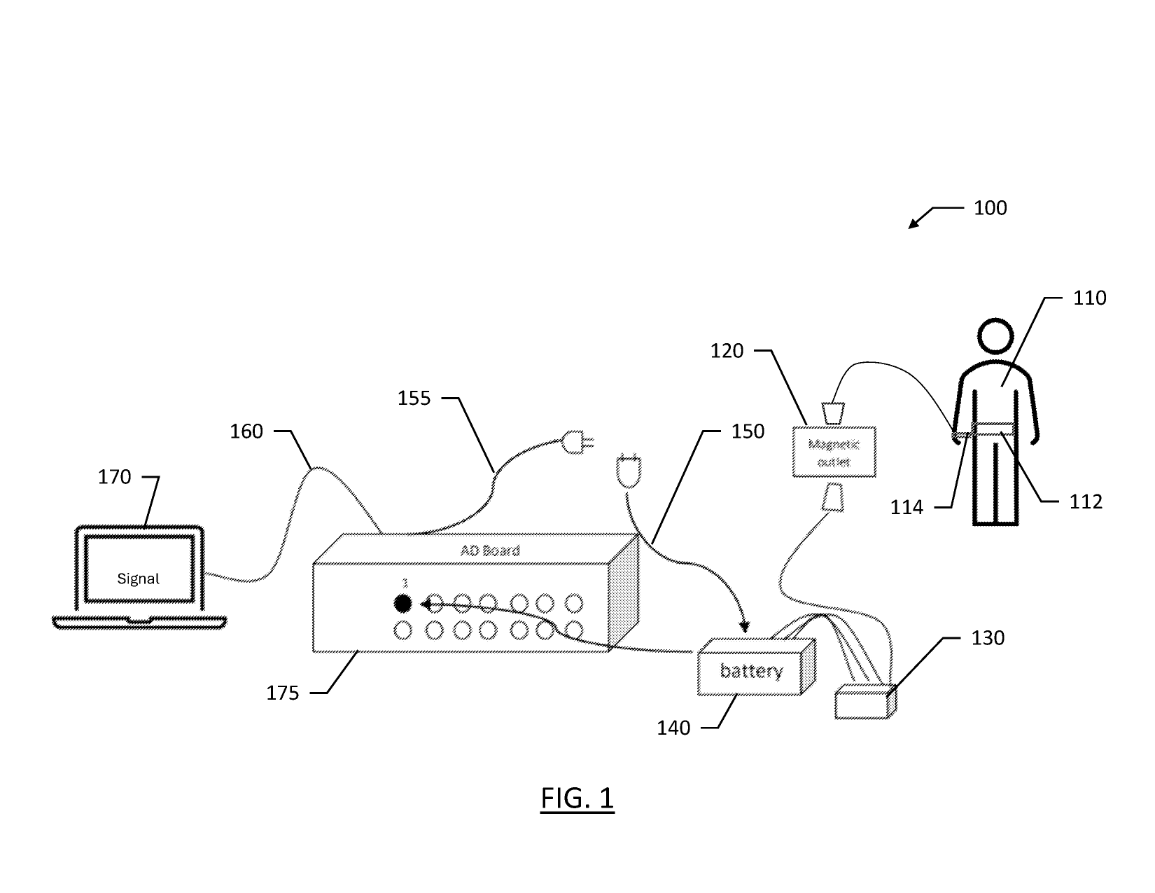

FIG. 1 illustrates an MR-compatible dynamometer system that is capable of measuring a degree of strain imparted by contraction of a muscle, specifically the shoulder. This MR-compatible dynamometer produces and measures isometric contractile force of the shoulder to enable viewing in real time by a medical professional for visual feedback. The device of example embodiments allows for functional MRI testing of muscles during MR procedures. As shown, the system 100 includes a patient 110 who wears the device described herein. The device includes a waist attachment 112 and a wrist attachment 114, as detailed further below. A non-ferrous load cell is connected between the waist attachment 112 and the wrist attachment 114.

The device is connected from the inside of the MRI chamber to the outside of the chamber via magnetic outlet 120 to a preamplifier 130, powered by battery 140. The battery 140 and an analog-to-digital conversion apparatus 175 are powered by power cables 150 and 155. A computer 170 is connected to the analog-to-digital conversion apparatus 175 by a cable 160 (e.g., a universal serial bus (USB) cable) or wirelessly in some embodiments.

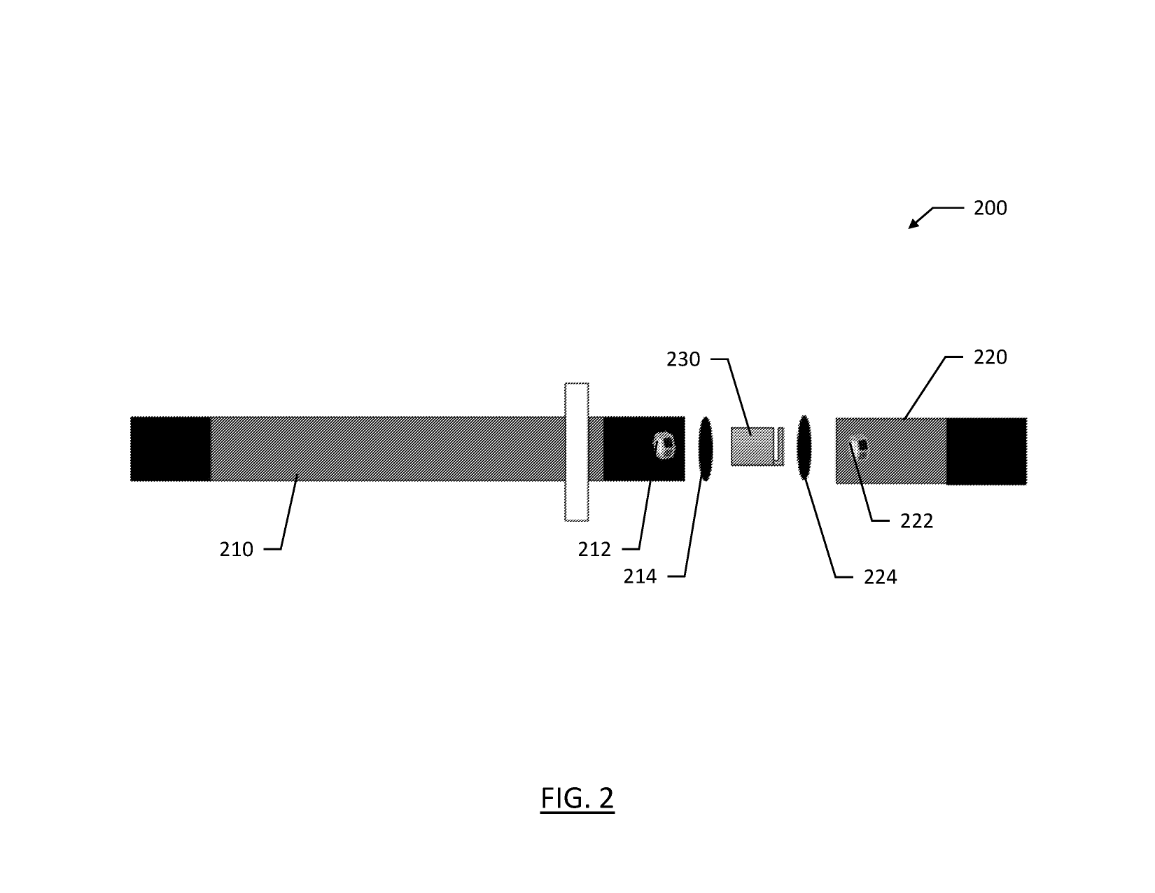

The device of example embodiments is illustrated in greater detail in FIG. 2. As shown, the device 200 includes several components. The depicted embodiment includes a first strap 210 and a second strap 220. These may be, for example, straps having Velcro® closures to secure around a patient. The first strap 210 is the larger of the two straps and is configured to be secured about the waist of a patient. The second strap 220 is configured to be secured about the wrist of a patient. With the patient in a position in which their arm is laying against their side, the first strap 210 is positioned on the waist to align with the second strap 220 positioned on the wrist. The straps may be adjustable in length to accommodate patients of various sizes.

The device 200 of FIG. 2 also includes a load cell 230, where the load cell is attached between the first strap 210 and the second strap 220, though shown in FIG. 2 separated for understanding. The load cell 230 can be secured to the first strap 210 using a fastener 212 and a washer 214 (e.g., a plastic or non-ferrous washer). Similarly, the second strap 220 can be secured to the load cell 230 using a fastener 222 and a washer 224. The fasteners may be releasably attached to the load cell to permit disassembly of the first strap 210 from the second strap 220, and separation and replacement (as needed) of the load cell 230.

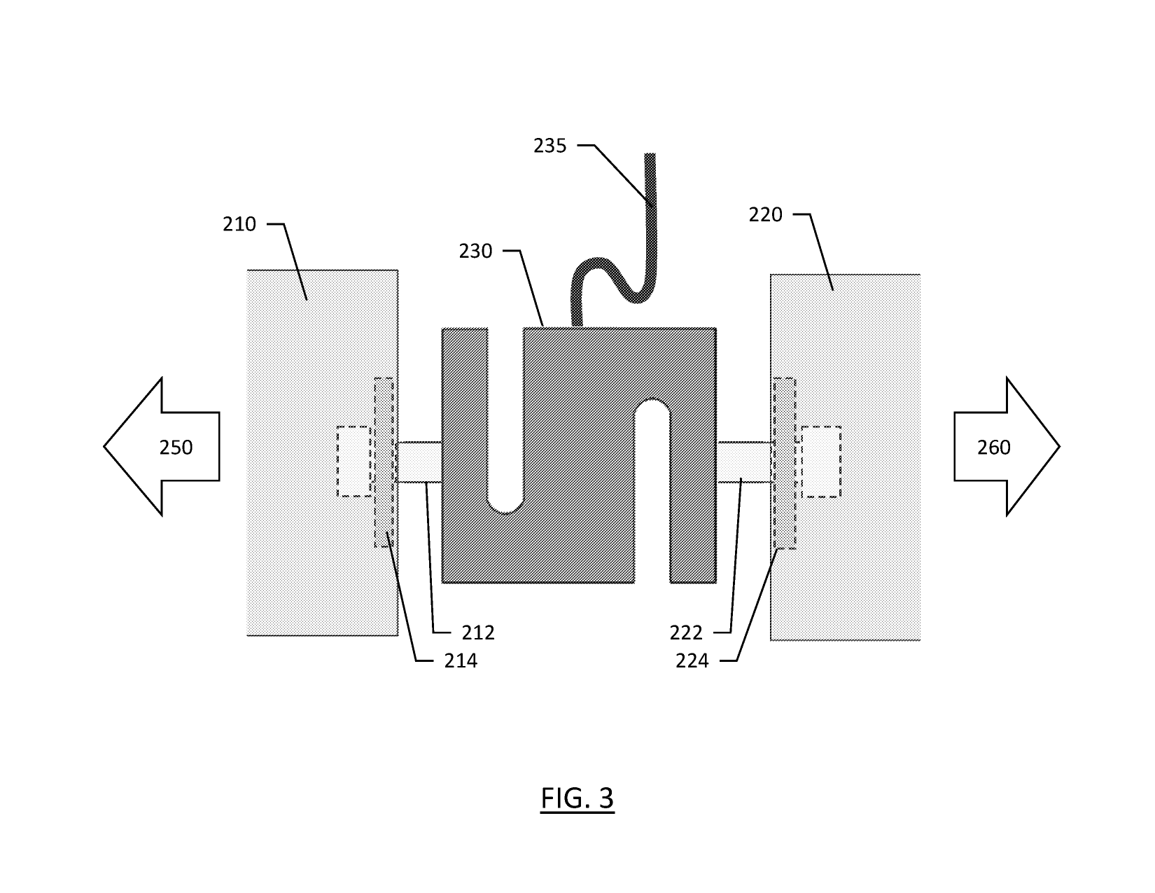

FIG. 3 illustrates a detail view of the load cell 230 as attached to the first strap 210 by the fastener 212 along with washer 214. An opposing side of the load cell 230 is attached to the second strap 220 by the fastener 222 along with washer 224. The fasteners permit relative rotation between the load cell 230 and the straps, while the washers preclude heads of the fasteners from being pulled through the material of the straps. The washers may be embedded within the strap or on an inside of the strap facing the wearer of the strap. In such a case, a head of the fastener may be rounded or flush with the washer (e.g., via chamfer in the washer) to prevent the fastener head from negatively impacting the wearer. The load cell 230 is placed in tension by relative motion by the first strap 210 and the second strap 220 moving away from one another. FIG. 3 illustrates this tension with the first strap 210 providing a force in the direction of arrow 250, while the second strap 220 provides a force in the direction of arrow 260. The load cell 230 provides an indication of the force, such as via wire 235, to the analog-to-digital conversion apparatus 175 as shown in FIG. 1, through the magnetic outlet and the preamplifier. The signal can then be read at computer 170 to understand the real time force between the first strap 210 and the second strap 220, corresponding to a movement of the arm away from the body of the patient.

While the attachment mechanisms to a patient are described herein as straps, embodiments can be attached to a person using other mechanisms. For example, a patient may wear a belt or harness about their waist, such that the attachment mechanism for the load cell may include a clip or other apparatus to connect the load cell to the belt.

The device 200 illustrated in FIGS. 2 and 3 and described above is compatible with magnetic resonance imaging, such that the device provides a magnetic resonance dynamometer that is able to provide real time feedback of the abduction force exerted between the patient's body and arm. This force, applied by muscles in the shoulder, provides the ability to perform a functional MRI while seeing the contractions of a muscle dynamically.

Embodiments described herein employ a shoulder device to develop a novel neuroimaging task that requires and measures precise shoulder force control. Outside the MRI scanner, the device 200 can be employed to determine a participant's maximal voluntary force for isometric shoulder abduction. This facilitates determination of force targets based on a percentage of each participant's maximal value, effectively normalizing the force task to each participant's strength. This is an important consideration particularly when working with individuals experiencing shoulder pain.

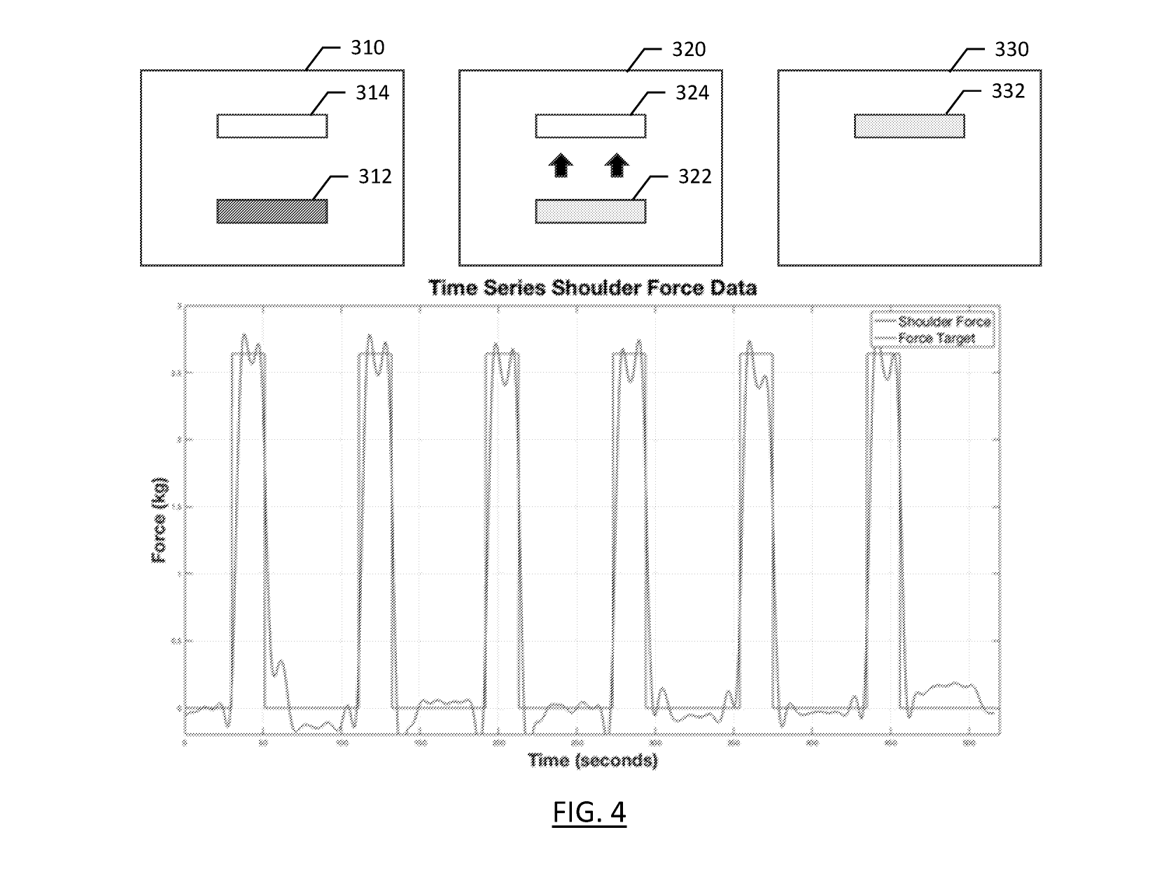

Inside the MRI scanner, a participant can be provided real-time visual feedback of a force target. FIG. 4 illustrates an example embodiment in which a display 310 presents a red rectangle 312 toward a bottom of the display indicates for the participant to rest while the white rectangle 314 is the target. A trial begins when the bottom rectangle turns green, shown in display 320 with green rectangle 322 and target white rectangle 324. The participant then generates shoulder force to elevate or lift the green rectangle 322 toward the target white rectangle 324. The goal is to have the green rectangle 322 move in the direction of the arrows on the display with the application of shoulder force. Display 330 illustrates when the shoulder force generated by the participant is in the target range and the green rectangle 332 overlies the target white rectangle indicate the proper shoulder force is being applied. The plot at the bottom of FIG. 4 illustrates the shoulder force as the curvilinear line, while the force target is depicted as the square wave. This plot presents a time series force tracing displaying the target force and the shoulder force generated by the participant. Using these methods, we can provide a patient-specific force target. Modeling force production enables measurement of brain activity associated with task performance. Shoulder motor control can be precisely quantified using the time series data.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An apparatus comprising:
a first strap configured to be attached to a patient proximate a first location;
a second strap configured to be attached to the patient proximate a second location; and
a load cell connected between the first strap and the second strap, wherein the load cell is configured to measure a force exerted between the first location of the patient and the second location of the patient.

2. The apparatus of claim 1, wherein the apparatus is magnetic resonance imaging compatible.

3. The apparatus of claim 1, wherein the load cell is magnetic resonance imaging compatible.

4. The apparatus of claim 1, wherein the first strap is configured to be attached to the patient proximate a waist of the patient, wherein the second strap is configured to be attached proximate a wrist of the patient.

5. The apparatus of claim 4, wherein a force exerted between the waist of the patient and the wrist of the patient comprises a contraction of at least one muscle in a shoulder associated with an intended abduction movement.

6. The apparatus of claim 1, wherein the load cell is connected to the first strap with a first fastener and to the second strap with a second fastener, wherein at least one of the first fastener and the second fastener are removably connected to the load cell.

7. The apparatus of claim 6, wherein the first fastener and the second fastener each permit rotation of the load cell relative to the first strap and the second strap.

8. The apparatus of claim 7, further comprising a first washer and a second washer, wherein the first strap is disposed between the load cell and the first washer and wherein the second strap is disposed between the load cell and the second washer.

9. A system comprising:
a magnetic resonance dynamometer;
an analog-to-digital converter; and
a computer, wherein the magnetic resonance dynamometer is configured to measure abduction force exerted by a patient during a magnetic resonance imaging procedure, and wherein the computer provides for a graphical indication of a force exerted in real time during the magnetic resonance imaging procedure.

10. The system of claim 9, wherein the magnetic resonance dynamometer is an upper extremity magnetic resonance dynamometer configured to measure the abduction force exerted by the patient from an upper extremity.

11. The system of claim 10, wherein the magnetic resonance dynamometer comprises:
a first strap configured to be attached to a patient proximate a first location;
a second strap configured to be attached to the patient proximate a second location; and
a load cell connected between the first strap and the second strap, wherein the load cell is configured to measure the force exerted between the first location of the patient and the second location of the patient.

12. The system of claim 11, wherein the magnetic resonance dynamometer is magnetic resonance imaging compatible.

13. The system of claim 11, wherein the load cell is magnetic resonance imaging compatible.

14. The system of claim 11, wherein the first strap is configured to be attached to the patient proximate a waist of the patient, wherein the second strap is configured to be attached proximate a wrist of the patient.

15. The system of claim 14, wherein the force exerted between the waist of the patient and the wrist of the patient comprises a contraction of at least one muscle in a shoulder associated with an intended movement.

16. The system of claim 11, wherein the load cell is connected to the first strap with a first fastener and to the second strap with a second fastener, wherein at least one of the first fastener and the second fastener are removably connected to the load cell.

17. The system of claim 16, wherein the first fastener and the second fastener each permit rotation of the load cell relative to the first strap and the second strap.

18. The system of claim 17, further comprising a first washer and a second washer, wherein the first strap is disposed between the load cell and the first washer and wherein the second strap is disposed between the load cell and the second washer.

19. The system of claim 9, further comprising:a display, wherein the display is configured to display a force signal as the patient exerts force in real time during the magnetic resonance imaging procedure.
